(19) United States
(12) Patent Application Publication
(10) Pub. No.: US 2024/0277218 A1
(43) Pub. Date: Aug. 22, 2024

(54) OPHTHALMIC ENDOSCOPE UTILIZING NEAR-INFRARED SPECTRUM

(71) Applicant: Alcon Inc., Fribourg (CH)

(72) Inventor: Paul R. HALLEN, Colleyville, TX (US)

(21) Appl. No.: 18/629,779

(22) Filed: Apr. 8, 2024

Related U.S. Application Data
(62) Division of application No. 17/454,160, filed on Nov. 9, 2021.
(60) Provisional application No. 63/114,039, filed on Nov. 16, 2020.

(51) Int. Cl. A61B 1/07; A61B 1/00; A61B 1/06; A61F 9/007

(52) U.S. Cl. CPC A61B 1/07; A61B 1/00045; A61B 1/0638; A61B 1/0661; A61F 9/00736; A61B 2562/0219; A61B 2562/028

(57) ABSTRACT

An ophthalmic endoscope includes a surgical handpiece and an endoscopic tip coupled to the surgical handpiece. A probe extends from the endoscopic tip. An illumination source is disposed in the surgical handpiece. A plurality of illumination fibers are disposed in the probe. The plurality of illumination fibers include a first end coupled to the illumination source and a second end that projects illumination outwardly from the probe. A wavelength of illumination supplied by the illumination source is adjustable between visible light and near-infrared light.

### PRIORITY CLAIM

This application is a divisional of U.S. patent application Ser. No. 17/454,160, filed Nov. 9, 2021, which claims the benefit of priority of U.S. Provisional Patent Application No. 63/114,039, filed on Nov. 16, 2020, which are hereby incorporated by reference in their entirety as though fully and completely set forth herein.

### TECHNICAL FIELD

The present disclosure relates generally to ophthalmic visualization and more particularly, but not by way of limitation, to utilization of the near-infrared spectrum for visualization of collector channels in treatment of glaucoma.

### BACKGROUND

This section provides background information to facilitate a better understanding of the various aspects of the disclosure. It should be understood that the statements in this section of this document are to be read in this light, and not as admissions of prior art.

Glaucoma is a serious ophthalmic condition that, if left untreated, can result in damage to the optic nerve leading to loss of visual field and eventual blindness. A major risk factor for many types of glaucoma is elevated intraocular pressure. Intraocular pressure is regulated by the production of aqueous humor by the ciliary processes of the eye and eventual drainage of the aqueous humor through the trabecular meshwork.

A number of surgical interventions are utilized in the treatment of glaucoma. These interventions include canaloplasty, trabeculectomy, and glaucoma drainage implants such as minimally-invasive glaucoma stent (MIGS) devices. Each of these interventions requires visualization of aqueous veins within the trabecular meshwork and schlemm's canal. Aqueous veins are generally not visible under the visible-light spectrum. The ability to visualize the aqueous veins during surgical intervention allows placement of implants near the aqueous veins, thereby increasing surgical efficacy.

### SUMMARY

Aspects of the disclosure relate to an ophthalmic endoscope. The ophthalmic endoscope includes a surgical handpiece and an endoscopic tip coupled to the surgical handpiece. A probe extends from the endoscopic tip. An illumination source is disposed in the surgical handpiece. A plurality of illumination fibers are disposed in the probe. The plurality of illumination fibers include a first end coupled to the illumination source and a second end that projects illumination outwardly from the probe. A wavelength of illumination supplied by the illumination source is adjustable between visible light and near-infrared light.

Aspects of the disclosure relate to an ophthalmic surgical system. The ophthalmic surgical system includes a surgical console, a processor disposed in the surgical console, and a display coupled to the surgical console. A surgical handpiece is coupled to the surgical console. The surgical handpiece includes a endoscopic tip. A probe extends from the endoscopic tip. An illumination source is disposed in the surgical handpiece. A plurality of illumination fibers are disposed in the probe. The plurality of illumination fibers include a first end coupled to the illumination source and a second end that projects illumination outwardly from the probe. A plurality of imaging fibers are disposed in the surgical handpiece. The plurality of imaging fibers include a first end that receives illumination from a surgical site and a second end coupled to an active-pixel sensor. The active-pixel sensor being electrically coupled to the processor. The surgical console facilitates selection of a wavelength of illumination supplied by the illumination source between visible light and near-infrared light.

Aspects of the disclosure relate to a method. The method includes inserting a probe into an ophthalmic incision. The probe is coupled to a surgical handpiece. A wavelength of illumination supplied by the illumination source is selected via the surgical console between visible light and near-infrared light. Illumination is supplied to the surgical site via a plurality of illumination fibers disposed in the probe. Illumination is supplied to an active-pixel sensor disposed in the surgical handpiece via a plurality of imaging fibers disposed in the probe. A signal is transmitted corresponding to an image of the surgical site from the active-pixel sensor to a process associated with the surgical console. The image is displayed on the surgical console.

This summary is provided to introduce a selection of concepts that are further described below in the detailed description. This summary is not intended to identify key or essential features of the claimed subject matter, nor is it intended to be used as an aid in limiting the scope of claimed subject matter.

### BRIEF DESCRIPTION OF THE DRAWINGS

The disclosure is best understood from the following detailed description when read with the accompanying figures. It is emphasized that, in accordance with standard practice in the industry, various features are not drawn to scale. In fact, the dimensions of various features may be arbitrarily increased or reduced for clarity of discussion.

FIG. 1 is a schematic diagram illustrating anatomical structures of the eye;

FIG. 2A is a schematic diagram illustrating a visualization system according to aspects of the disclosure;

FIG. 2B is an illustration of an image displayed on a surgical console showing an orientation arrow according to aspects of the disclosure;

FIG. 3 is a side view of an endoscopic tip according to aspects of the disclosure;

FIG. 4 is an enlarged cross-sectional view of an endoscopic tip illustrating illumination and imaging fibers contained therein according to aspects of the disclosure; and

FIG. 5 is a flow diagram illustrating a process for visualizing a surgical site according to aspects of the disclosure.

### DETAILED DESCRIPTION

Various embodiments will now be described more fully with reference to the accompanying drawings. The disclosure may, however, be embodied in many different forms and should not be construed as limited to the embodiments set forth herein.

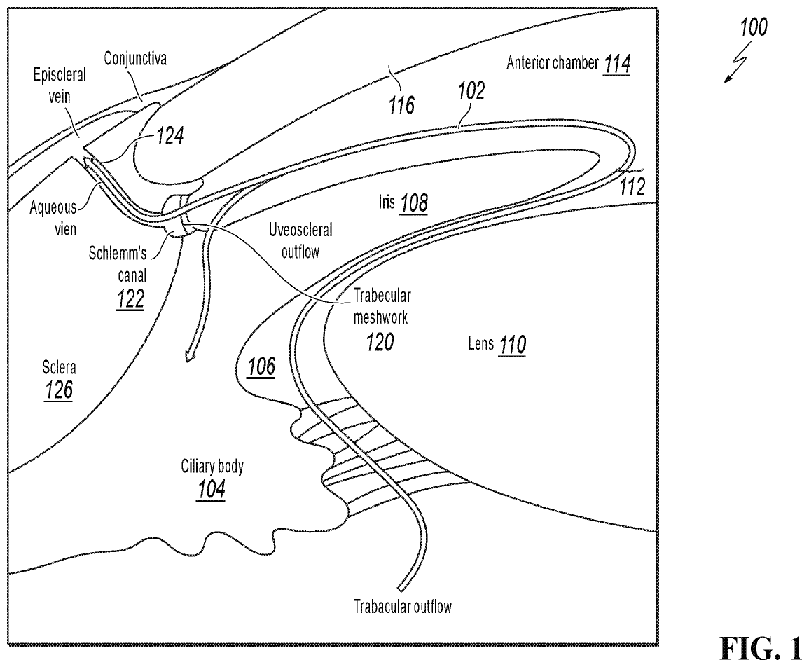

FIG. 1 is a schematic diagram illustrating anatomical structures of an eye 100. Aqueous humor (depicted by arrows 102) is produced in the ciliary processes 104 and enters the posterior chamber 106. The posterior chamber is bounded anteriorly by the iris 108 and posteriorly by the lens 110. The aqueous humor 102 travels through the pupil 112 in the anterior chamber 114. The anterior chamber 114 is bounded anteriorly by the cornea 116 and posteriorly by the iris 108. The aqueous humor 102 then exits the anterior chamber 114 through the trabecular meshwork 120 and enters the Schlemm's canal 122. From the Schlemm's canal 122, the aqueous humor travel through a plurality of aqueous veins 124 formed through the sclera 126.

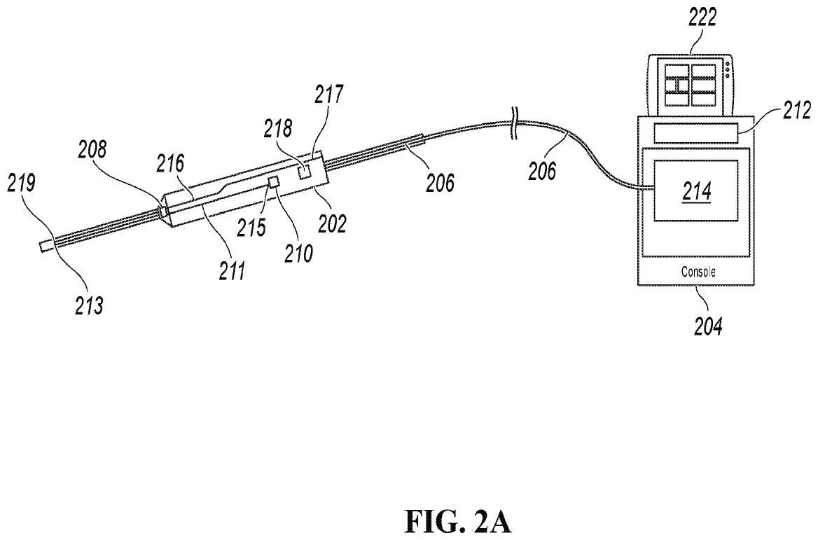

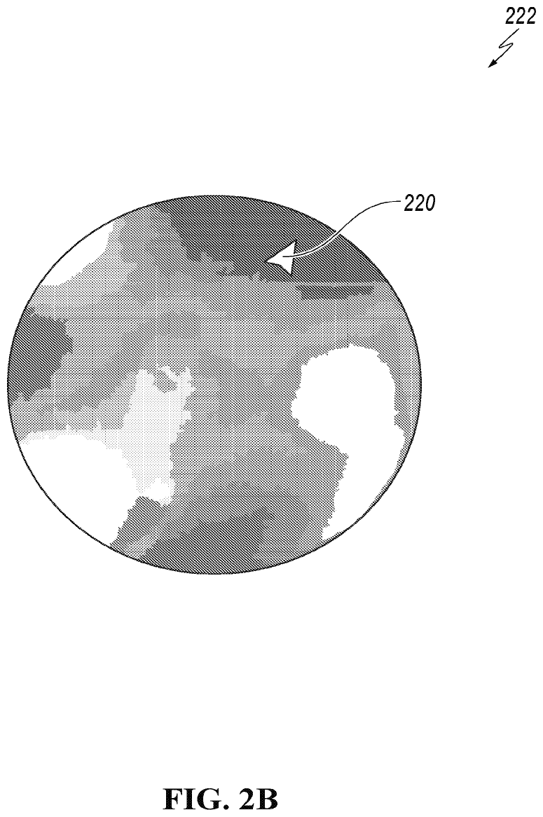

FIG. 2A is a schematic diagram illustrating a visualization system 200. The visualization system 200 includes a endoscopic handpiece 202, which is operatively coupled to a surgical console 204. In various embodiments, the surgical console 204 provides a number of functions for ophthalmic surgical interventions including, for example, supply of irrigation fluid to a surgical site, supply of suction to the surgical site for aspiration of fluids and tissue, storage of aspirated fluids and tissue, and supply of illumination for visualization of the anatomical structures of the eye 100. A display 222 is included with the surgical console 222. The surgical console 204 includes an illumination source 214. In various embodiments, the surgical console 204 allows selection of illumination wavelength. In various embodiments, a wavelength of illumination supplied by the illumination source 214 may be varied between, for example, visible light having a wavelength of approximately 400 nm to approximately 700 nm to near-infrared light having a wavelength of approximately 1 μm to approximately 10 μm. In various embodiments, the surgical console 204 allows selection of illumination intensity as well as color balance (known as red/blue/green or “RBG” control). In such embodiments, the wavelength may be selected between visible light or near-infrared depending on the structures being visualized as well as the preferences of the surgeon. Illumination is supplied to the endoscopic handpiece 202 via a conductive cable 206. In various embodiments, the cable 206 is capable of transmitting electrical and optical signals between the surgical console 204 and the handpiece 202.

Still referring to FIG. 2A, the surgical handpiece 202 is coupled to an endoscopic tip 208. The surgical handpiece 202 contains an active-pixel sensor 210 such as, for example, a complementary metal-oxide semiconductor (CMOS) sensor. The active-pixel sensor 210 receives light reflected from an interior of the surgical site via a plurality of imaging fibers 211 and converts individual pixels into a digital signal corresponding to the image. The plurality of imaging fibers 211 include a first end 213 that receives illumination reflected from the surgical site and a second end 215 that is coupled to the active-pixel sensor 210. This digital signal is then transmitted to a processor 212 disposed in the surgical console 204 via the cable 206. The processor 212 may be any microprocessor, microcontroller, programmable element, or other device or collection of devices for processing instructions for the control of the surgical handpiece 202, or the surgical console 204. The illumination source 214 provides illumination to the handpiece 202 and a plurality of illumination fibers 216 via the cable 206. The illumination fibers 216 transmit illumination to the surgical site. The plurality of illumination fibers include a first end 217 coupled to the cable 206 and a second end 219 that projects illumination outwardly from the endoscopic tip 208.

Still referring to FIG. 2A, the surgical handpiece includes a gyroscopic chip 218. In various embodiments, the gyroscopic chip 218 may be, for example, a microelectromechanical systems (MEMS) three-axis gyroscope or other type of gyroscopic chip. During operation, the gyroscopic chip facilitates orientation and stabilizing of the image captured by the active-pixel sensor 210 that is transmitted to, and displayed by, the display 222 of the surgical console 204. In various embodiments, the gyroscopic chip mitigates incidental rotational movement of the surgical handpiece 202 during the course of the surgical intervention and stabilizes the image displayed on the display 222 of the surgical console 204. FIG. 2B is an illustration of an image displayed on the display 222 of the surgical console 204. In various embodiments, an orientation arrow 220 is placed on the image to facilitate orientation of the image.

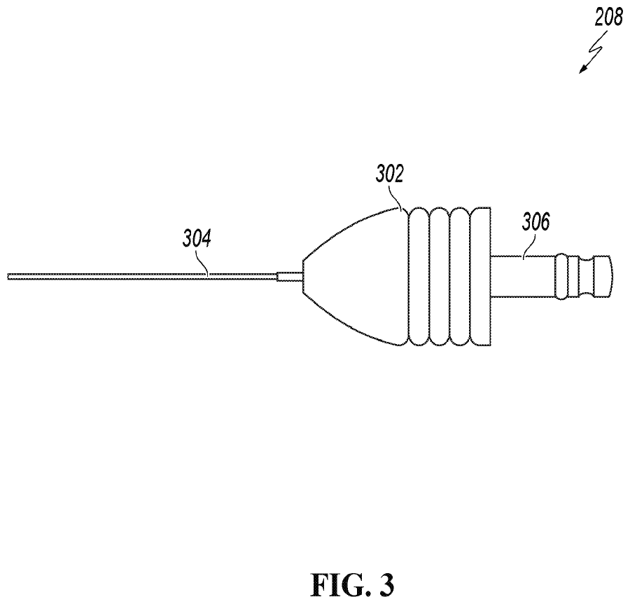

FIG. 3 is a side view of the endoscopic tip 208. The endoscopic tip 208 includes a hub 302 and a probe 304. The hub 302 includes a connector 306 that engages with the surgical handpiece 202 and facilitates transmission of optical and electrical signals between the endoscopic tip 208 and the surgical handpiece 202. During use, the probe 304 is inserted into an incision at a surgical site. The illumination fibers 216 transmit illumination from the illumination source 214 to the surgical site. The imaging fibers 211 transmit illumination reflected from the surgical site to the active-pixel sensor 210. Use of near-infrared illumination enables visualization of structures such as the trabecular meshwork 120 and aqueous veins 124. Such illumination facilitates accurate placement of glaucoma drainage implants such as, for example, MIGS devices or other devices used in the treatment of, for example, glaucoma.

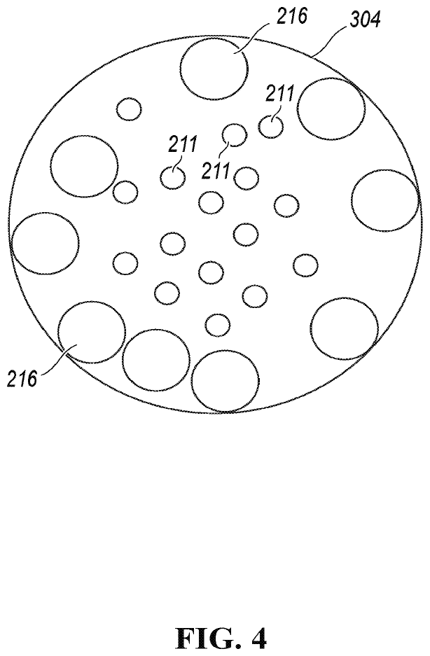

FIG. 4 is an enlarged cross-sectional view of the probe 304 illustrating illumination fibers 216 and imaging fibers 211. The illumination fibers 216 and the imaging fibers 211 are disposed in the probe 302 such that the illumination fibers 216 and the imaging fibers 211 are substantially parallel to a long axis of the probe. In various embodiments, the illumination fibers 216 have a cross-sectional diameter that is larger than the cross-sectional diameter of the imaging fibers 211. In various embodiments, the illumination fibers 216 may have a cross-sectional diameter that is, for example, 3 to 5 times greater than the cross-sectional diameter of the imaging fibers 211. In other embodiments, the illumination fibers 216 have a cross-sectional diameter that is approximately 10 times greater than the cross-sectional diameter of the imaging fibers. In a typical embodiment, approximately 30 to approximately 40 illumination fibers 216 are positioned around an interior perimeter of the probe 304 while approximately 30,000 imaging fibers 211 are positioned near a center of the probe 304.

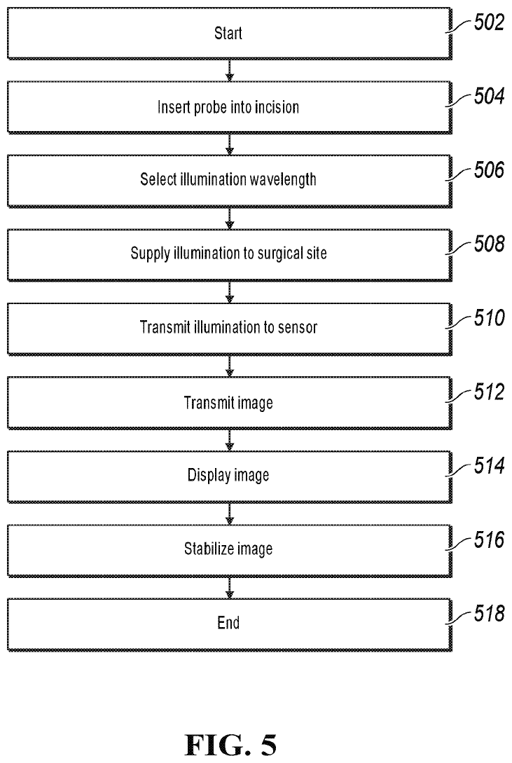

FIG. 5 is a flow diagram of a process 500 for visualizing a surgical site. The process 500 begins at block 502. At block 504, the probe 304 is inserted into an incision. In various embodiments, the incision be, for example, a cataract incision or other type of incision. At block 506, an illumination wavelength, intensity, and color balance is selected on the surgical console 204. In various embodiments, the illumination wavelength may be for example, visible light, near-infrared light, or other appropriate wavelength depending on the anatomical structures being visualized and the nature of the surgical intervention. By way of example, during glaucoma surgery, the aqueous veins 124 are frequently not visible when illuminated with visible light. The aqueous veins 124 become visible under visualization with near-infrared illumination, thereby facilitating placement of, for example, MIGS devices or other devices that may be used in the treatment of glaucoma. At block 508, illumination is supplied to the surgical site via the illumination fibers 216. At block 510, illumination is reflected from the surgical site and transmitted, via the plurality of imaging fibers 211, to the active-pixel sensor 210 so as to capture an image of the surgical site. At block 512, the active-pixel sensor 210 transmits an electrical signal corresponding to the captured image to the surgical console 204. At block 514, the surgical console 204 displays the image captured by the active-pixel sensor 210 on the display 222. At block 516, during surgery, the gyroscopic chip 218 stabilizes the image displayed on the surgical console 204 and prevents incidental movement of the displayed image due to incidental movement of the surgical handpiece 202. The process 500 ends at block 518.

The term “substantially” is defined as largely but not necessarily wholly what is specified (and includes what is specified; e.g., substantially 90 degrees includes 90 degrees and substantially parallel includes parallel), as understood by a person of ordinary skill in the art. In any disclosed embodiment, the terms “substantially,” “approximately,” “generally,” and “about” may be substituted with “within [a percentage] of” what is specified.

Conditional language used herein, such as, among others, “can,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

While the above detailed description has shown, described, and pointed out novel features as applied to various embodiments, it will be understood that various omissions, substitutions, and changes in the form and details of the devices illustrated can be made without departing from the spirit of the disclosure. As will be recognized, the processes described herein can be embodied within a form that does not provide all of the features and benefits set forth herein, as some features can be used or practiced separately from others. The scope of protection is defined by the appended claims rather than by the foregoing description. All changes which come within the meaning and range of equivalency of the claims are to be embraced within their scope.

1. A method, comprising:
inserting a probe into an ophthalmic incision, the probe being coupled to a surgical handpiece;
selecting, via a surgical console, a wavelength of illumination light supplied by an illumination source between visible light and near-infrared light;
supplying the illumination light to a surgical site via a plurality of illumination fibers disposed in the probe;
receiving reflected illumination light at an active-pixel sensor disposed in the surgical handpiece via a plurality of imaging fibers disposed in the probe;
transmitting a signal corresponding to an image of the surgical site from the active-pixel sensor to a processor associated with the surgical console; and
displaying the image on the surgical console.

2. The method of claim 1, further comprising supplying near-infrared illumination light to the surgical site to visualize aqueous veins of an eye.

3. The method of claim 2, further comprising placing a stent device in the surgical site while supplying the near-infrared illumination light.

4. The method of claim 3, further comprising stabilizing, via a gyroscopic chip, the image displayed on the surgical console against incidental movement of the surgical handpiece.

5. The method of claim 4, comprising orienting, via the gyroscopic chip, the image displayed on the surgical console.

6. The method of claim 1, wherein the active-pixel sensor is a complementary metal-oxide semiconductor (CMOS) sensor.

7. The method of claim 1, wherein the incision is a cataract incision.

8. A method for glaucoma treatment, comprising:
inserting a probe into an incision in an eye, the probe being coupled to a surgical handpiece;
selecting, via a surgical console, a first wavelength of an illumination light supplied by an illumination source, the first wavelength comprising visible wavelength;
supplying the illumination light in the first wavelength to a surgical site in the eye via a plurality of illumination fibers disposed in the probe;
switching, via the surgical console, between the first wavelength of the illumination light to a second wavelength of the illumination light, the second wavelength comprising a near-infrared wavelength;
supplying the illumination light in the second wavelength to the surgical site via the plurality of illumination fibers disposed in the probe;
transmitting a signal corresponding to an image of the surgical site from the surgical handpiece to the surgical console via a plurality of imaging fibers disposed in the probe;
identifying, while supplying the illumination light in the second wavelength to the surgical site, aqueous veins at the surgical site; and
implanting, into the surgical site, a drainage device.

9. The method of claim 8, wherein the illumination light is supplied to an active-pixel sensor disposed in the surgical handpiece via the plurality of imaging fibers disposed in the probe.

10. The method of claim 9, wherein the active-pixel sensor is a complementary metal-oxide semiconductor (CMOS) sensor.

11. The method of claim 9, wherein the transmitting the signal corresponding to an image of the surgical site comprises transmitting the signal from the active pixel sensor to a processor associated with the surgical console.

12. The method of claim 8, further comprising displaying the image on a surgical console.

13. The method of claim 8, wherein the drainage device comprises a microinvasive glaucoma surgery device or a stent.

14. The method of claim 8, further comprising stabilizing, via a gyroscopic chip, the image displayed on the surgical console against incidental movement of the surgical handpiece.

15. The method of claim 14, comprising orienting, via the gyroscopic chip, the image displayed on the surgical console.

16. The method of claim 8, wherein the incision in the eye is a cataract incision.

17. A method, comprising:
inserting a probe in an incision in an eye, the probe being coupled to a surgical handpiece;
selecting, via a surgical console, a wavelength of illumination supplied by an illumination source between visible light and near infrared light, wherein the wavelengths of the visible light supplied by the illumination source range from 400 nanometers to 700 nanometers and the wavelengths of the near-infrared light supplied by the illumination source range from 1 micrometer to 10 micrometers;
supplying the illumination light to a surgical site via a plurality of illumination fibers disposed in the probe;
receiving reflected illumination light at an active-pixel sensor disposed in the surgical handpiece via a plurality of imaging fibers disposed in the probe;
transmitting a signal corresponding to an image of the surgical site from the active-pixel sensor to a processor associated with the surgical console; and
displaying the image on the surgical console.

18. The method of claim 17, wherein the plurality of imaging fibers comprise a first end coupled to the active pixel sensor in the surgical handpiece and a second end that receives reflected illumination light at a distal end of the probe, each of the plurality of imaging fibers having a first cross-sectional dimension.

19. The method of claim 18, wherein the plurality of illumination fibers comprise a first end configured to be coupled to an illumination source through a proximal end of the surgical handpiece and a second end that projects illumination light outwardly from the probe, each of the plurality of illumination fibers having a second cross-sectional dimension different than the first cross-sectional dimension.

20. The method of claim 17, further comprising supplying near-infrared illumination light to the surgical site to visualize aqueous veins of an eye; and
placing a drainage device in the surgical site while supplying the near-infrared illumination light.
